(19) United States
(12) Patent Application Publication
(10) Pub. No.: US 2021/0069286 A1
(43) Pub. Date: Mar. 11, 2021

(54) COMPOSITIONS AND METHODS FOR TREATING OR PREVENTING HYPERGLYCEMIA, INSULIN RESISTANCE, AND ASSOCIATED ORGAN DAMAGE

(71) Applicant: 9 Meters Biopharma, Inc., Raleigh, NC (US)

(72) Inventors: Christopher PRIOR, Raleigh, NC (US); Sandeep LAUMAS, Raleigh, NC (US)

(21) Appl. No.: 17/046,013

(22) PCT Filed: Apr. 8, 2019
(86) PCT No.: PCT/US2019/026277

§ 371 Date: Oct. 8, 2020

Related U.S. Application Data
(60) Provisional application No. 62/654,927, filed on Apr. 9, 2018.

(51) Int. Cl. A61K 38/08; A61K 9/00; A61K 38/17; A61K 31/155; A61K 31/64; A61K 38/28; A61K 31/426; A61K 47/64; A61K 47/68

(52) U.S. Cl. CPC A61K 38/08; A61K 9/0053; A61K 38/1796; A61K 31/155; A61K 47/644; A61K 38/28; A61K 31/426; A61K 47/643; A61K 47/68; A61K 31/64

(57) ABSTRACT

The present invention provides compositions and methods for treating hyperglycemia, insulin resistance, and associated organ damage, including in some embodiments diabetes mellitus (type 1 or 2), metabolic syndrome, obesity, fatty liver diseases, or kidney disease. In various embodiments, the invention involves administering a regimen of larazotide (or a derivative of larazotide) to a subject. In various embodiments, the regimen reduces dysfunction of the gastrointestinal epithelial barrier, thereby improving glycemic control. In various embodiments, the regimen of larazotide improves the effectiveness of conventional pharmaceutical interventions for hyperglycemia or diabetes mellitus.

### FIELD OF THE INVENTION

The present invention provides compositions and methods for treating patients exhibiting hyperglycemia or insulin resistance or associated conditions, including patients experiencing or at risk of organ damage.

### CROSS-REFERENCE TO RELATED APPLICATIONS

This application claims the benefit of U.S. Provisional Application No. 62/654,927, filed on Apr. 9, 2018, the entire contents of which are incorporated herein.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (Filename: “INN-019PC_ST25.txt”; Date created: Mar. 28, 2019; File size: 1.04 KB).

### BACKGROUND

Hyperglycemia, such as that associated with metabolic syndrome or diabetes mellitus type 2, can lead to a number of severe health impairments including damage to blood vessels and organs. Hyperglycemia is typically treated with agents such as metformin, basal insulin, GLP-1 receptor agonists, among others. However, disease control can remain elusive for many patients.

Accordingly, there remains a need for effective therapies for treating conditions such as hyperglycemia, insulin resistance, and metabolic syndrome, and particularly for patients whose disease is not adequately controlled by traditional pharmaceutical intervention, and who are at risk for damage or loss of function of vital organs such as the liver and kidneys. There remains a need to better manage the disease in many patients, and/or slow the progression of disease, including by preserving organ function and reducing organ damage.

The present invention addresses these and other objectives.

### SUMMARY OF THE INVENTION

The present invention provides compositions and methods for treating hyperglycemia, including in some embodiments insulin resistance, diabetes mellitus (type 1 or 2), metabolic syndrome, or obesity. In various embodiments, the invention involves administering a regimen of larazotide (or a derivative of larazotide) to a subject. In various embodiments, the regimen reduces dysfunction of the gastrointestinal epithelial barrier, thereby improving glycemic control. In various embodiments, the regimen of larazotide improves the effectiveness of conventional pharmaceutical interventions, such as metformin, basal insulin, glucose-dependent insulinotropic polypeptide (GIP) and/or GLP-1 receptor agonists, sulphonylurea, pPAR-gamma agonists, among others. In accordance with the invention, the larazotide regimen prevents complications of hyperglycemia, including cardiovascular complications and damage to organs. In some embodiments, the subject is exhibiting symptoms of liver or kidney disease, or is at risk of liver or kidney disease. For example, in embodiments, the invention ameliorates, prevents, or slows progression of inflammatory liver diseases such as non-alcoholic fatty acid liver disease (NAFLD), and non-alcoholic steatohepatitis (NASH). In these or other embodiments, the invention ameliorates, prevents, or slows progression of kidney disease, such as diabetic nephropathy.

Larazotide is a peptide agent that promotes tight junction integrity and reduced epithelial permeability, including in the gastrointestinal tract (GI). Larazotide has the amino acid sequence: Gly Gly Val Leu Val Gln Pro Gly (SEQ ID NO:1), and can be formulated for systemic or targeted release in portions of the GI (e.g., stomach, small intestine and/or large intestine). In some embodiments, the patient receives a larazotide derivative, for example, having one or more amino acid substitutions, deletions, and/or insertions with respect to SEQ ID NO:1.

In various embodiments, the present invention provides pharmaceutical compositions comprising the larazotide or derivative that are co-formulated with other therapeutic agents for the treatment of diabetes mellitus and which are suitable for oral delivery. For example, in some embodiments, larazotide or derivative is co-formulated with GLP-1 receptor agonist, dual GLP-1/GIP receptor agonist (e.g., LY3298176), insulin, metformin, sulphonylurea, pPAR-gamma agonist, meglitinide, α-glucosidase inhibitors, or thiazolidinediones.

In various embodiments, larazotide is formulated as a plurality of particles that release larazotide at different times in intestinal fluid, or at different locations in the intestine. In other embodiments, the formulation releases larazotide in a form that provides for a local sustained release at one or more locations, including sustained release from particles, gels, emulsions, or biodegradable matrix.

The present invention also provides for compositions that release multiple doses of the larazotide or derivative along the gastrointestinal tract. The overall release profile of such a formulation may be adjusted using, for example, multiple particle types or multiple layers. For example, in one embodiment, a first dose of the larazotide or derivative may be formulated for release in, for example, the small intestine (e.g., one or more of duodenum, jejunum, ileum), whereas a second dose is formulated for delayed release in, for example, the large intestines (e.g., one or more of cecum, ascending, transverse, descending or sigmoid portions of the colon, and rectum). In various embodiments, the composition and/or formulation may release at least three doses, at least four doses, or at least five doses of the larazotide or derivative at different locations along the intestines, at different times, and/or at different pH. In these embodiments, a dose of larazotide may be from 0.1 to 1 mg of larazotide, for example.

In accordance with certain embodiments of the invention, larazotide or derivative is administered more than once daily to promote GI tight junction integrity. For example, larazotide or derivative may be administered about two times daily, about three times daily, about four times daily, or about five times daily.

In some embodiments, larazotide or derivative thereof is heterologously expressed in a microorganism, which is administered to the patient as a probiotic therapy. In these embodiments, the invention provides broad coverage of the gastrointestinal tract with sustained release of larazotide for a period-of-time. Such larazotide producing probiotics can provide for an efficient and patient friendly therapy for such severe diseases that may otherwise involve organ damage and decline.

Other aspects and embodiments of the invention will be apparent from the following detailed description.

### DETAILED DESCRIPTION

The present invention provides compositions and methods for treating hyperglycemia, including in some embodiments insulin resistance, diabetes mellitus (type 1 or 2), metabolic syndrome, or obesity. In various embodiments, the invention involves administering a regimen of larazotide (or a derivative of larazotide) to a subject. In various embodiments, the regimen reduces dysfunction of the gastrointestinal epithelial barrier, thereby improving glycemic control and preventing or slowing the progression of associated organ injury. In various embodiments, the regimen of larazotide improves the effectiveness of conventional pharmaceutical interventions, such as metformin, basal insulin, GLP-1 receptor agonists, dual GLP-1/GIP receptor agonist (e.g., LY3298176), sulphonylurea, GIP receptor antagonists, pPAR-gamma agonists, among others. In accordance with the invention, the larazotide regimen prevents complications of hyperglycemia, including cardiovascular complications and damage to organs. For example, in embodiments, the invention ameliorates or prevents inflammatory liver diseases such as non-alcoholic fatty acid liver disease (NAFLD), non-alcoholic steatohepatitis (NASH), and cirrhosis. In these or other embodiments, the invention ameliorates, prevents, or slows progression of kidney disease, such as diabetic nephropathy and chronic kidney disease. In some embodiments, the invention ameliorates, prevents, or slows progression of hepatorenal syndrome.

Without wishing to be bound by any one theory, it is proposed that uncontrolled hyperglycemia can result in or be associated with intestinal barrier dysfunction. This dysfunction reduces the efficacy of traditional pharmaceutical interventions for hyperglycemia and diabetes, and allows for the diffusion of microbes and toxins (e.g., lipopolysaccharides or LPS) from the lumen of the intestine into the intestinal lamina propria and systemic circulation, which in turn cause systemic infection or damage to tissues and organs. Further, in subjects with hyperglycemia-induced intestinal barrier dysfunction, glucose may leak into the circulation via the disrupted epithelial tight junctions, which may impact the efficacy of pharmaceutical agents prescribed for glycemic control.

In some embodiments, the hyperglycemic patient has a consistent blood sugar level greater than about 5 mmol/l, or in some embodiments, greater than about 7 mmol/l, or greater than about 10 mmol/l, or greater than about 12 mmol/l or greater than about 15 mmol/l. In some embodiments, the patient is prediabetic, or has type 1 or type 2 diabetes. In some embodiments, the patient is undergoing therapy with one or more agents for glycemic control, including those selected from metformin, basal insulin, GLP-1 receptor agonists, dual GLP-1/GIP receptor agonist (e.g., LY3298176), sulphonylurea, meglitinide, pPAR-gamma agonist, α-glucosidase inhibitor, GIP receptor antagonists, and thiazolidinediones. In some embodiments, despite these therapies, the patient remains consistently hyperglycemic.

In certain embodiments, the patient is overweight or obese. For example, the patient in some embodiments has a body mass index of from 25 to 30, or a body mass index of at least 30. In some embodiments, the patient has a body mass index of at least 35. Body mass index or BMI is a measure of body fat based on height and weight, and the determination of BMI is well known.

In embodiments of the invention, the patient has metabolic disease. Metabolic disease may be defined by the presence in the patient of at least two of: (1) triglycerides>150 mg/dL (1.7 mmol/L); (2) HDL cholesterol<40 mg/dL (1.03 mmol/L) for a male, <50 mg/dL (1.29 mmol/L) for a female; (3) systolic BP>130 or diastolic BP>85 mm Hg, or the patient is being treated for hypertension; (4) fasting plasma glucose (FPG)>100 mg/dL (5.6 mmol/L) or the patient is being treated for hyperglycemia; and (5) an elevated waist circumference equal to or greater than 40 inches (102 cm) for men, or equal to or greater than 35 inches (88 cm) for women.

In some embodiments, the patient may further exhibit complications from hyperglycemia, such as inflammatory liver disease, fatty liver disease, including, but not limited to non-alcoholic fatty acid liver disease (NAFLD), non-alcoholic steatohepatitis (NASH), or cirrhosis. In these or other embodiments, the patient exhibits kidney disease, such as diabetic nephropathy or chronic kidney disease. In some embodiments, the patient has or exhibits signs of hepatorenal syndrome. In some embodiments, the patient exhibits pancreatitis, adipose tissue inflammation, atherosclerosis, and/or neurodegeneration.

In various embodiments, the present invention provides for administering larazotide or derivative in combination with other therapeutic agents for the treatment and/or prevention of chronic inflammatory disease, including complications and conditions associated with loss of renal function, hyperglycemia, or diabetes. For example, in some embodiments, larazotide or derivative is administered in combination with one or more of metformin, basal insulin, GLP-1 receptor agonists, sulphonylurea, pPAR-gamma agonists, and sodium butyrate.

In various embodiments, the present invention provides compositions and methods for treating or preventing complications and conditions associated with loss of renal function. For example, in some embodiments, the regimen of larazotide or derivative improves kidney and/or liver function, or slows decline of liver or kidney function. In some embodiments, insulin sensitivity is improved, and/or cholesterol levels are reduced. In accordance with the invention, the larazotide regimen prevents, reduces, ameliorates, or slows progression and/or severity of complications and conditions associated with loss of renal function, including in some embodiments hepatorenal syndrome, inflammatory liver condition (e.g., NAFLD, NASH, ASH, cirrhosis, and/or hepatitis), diabetes (Type 1 or Type 2), hyperglycemia, and hypercholesterolemia. In embodiments, the invention results in improved kidney and/or liver function.

Without wishing to be bound by any one theory, it is proposed that uncontrolled uremia due to loss of renal function can result in or be associated with intestinal barrier dysfunction. The resulting tissue injury stimulates the influx of inflammatory leukocytes, which can further exacerbate the loss of epithelial barrier function. This dysfunction impacts liver and kidney function, due in part to the introduction of microbial toxins into the circulation, and can impact the efficacy of traditional chronic disease management strategies. For example, the diffusion of microbes and toxins (e.g., lipopolysaccharides or LPS) from the lumen of the intestine into the intestinal lamina propria and systemic circulation can cause systemic infection or damage to tissues and organs, including but not limited to the liver. In turn, liver disease can cause physiological changes (including hypertension) affecting the circulation and kidney perfusion, for example, thus exacerbating loss of kidney function. Administration of larazotide according to this disclosure interrupts this cycle of organ damage and GI epithelial permeability.

In some embodiments, the subject presents with loss of renal function and exhibits abnormally high levels of creatine in the blood, or protein or red blood cells in the urine. Indeed, creatine in the blood is indicative of low glomerular filtration rate, a hallmark of loss of kidney function. In some embodiments, serum creatinine levels are affected by degree of liver cirrhosis, hyperbilirubinemia, and nutrition. In some embodiments, the subject is undergoing therapy with one or more agents for managing chronic kidney disease progression and/or symptoms, including, but not limited to, high blood pressure medications, medications to lower cholesterol levels, medications to treat anemia, medications to relieve swelling, calcium or vitamin D supplements, and sodium butyrate.

In an embodiment, the subject has chronic kidney disease, which can be Stage 1, Stage 2, Stage 3, or Stage 4. Kidney disease can manifest from small changes in kidney function, resulting in low glomerular filtration rate (GFR) measured in ml/min/1.73 m², which can eventually lead to loss of kidney function and/or kidney failure. In some embodiments, patients with Stage 1 chronic kidney disease have a GFR of 90 ml/min/1.73 m² or higher, indicating kidney damage with normal kidney function; patients with Stage 2 chronic kidney disease have a GFR of 89 to 60 ml/min/1.73 m², indicating kidney damage with mild loss of kidney function; patients with Stage 3a chronic kidney disease have a GFR of 59 to 45 ml/min/1.73 m², indicating mild to moderate loss of kidney function; patients with Stage 3b chronic kidney disease have a GFR of 44 to 30 ml/min/1.73 m², indicating moderate to severe loss of kidney function; patients with Stage 4 chronic kidney disease have a GFR of 29 to 15 ml/min/1.73 m², indicating severe loss of kidney function; and patients with Stage 5 chronic kidney disease have a GFR of less than 15 ml/min/1.73 m², indicating total loss of kidney function and/or kidney failure. In further embodiments, patients with chronic kidney disease exhibit high levels of serum creatinine.

In various embodiments, the subject may have a fatty liver disease including, but not limited to non-alcoholic fatty acid liver disease (NAFLD), non-alcoholic steatohepatitis (NASH), or a fatty liver disease resulting from hepatitis, obesity, diabetes, insulin resistance, hypertriglyceridemia, abetalipoproteinemia, glycogen storage disease, Weber-Christian disease, Wolmans disease, acute fatty liver of pregnancy, and lipodystrophy. In some embodiments, improvements in intestinal barrier function limit the amount of toxins such as LPS that enter circulation and which can ultimately exacerbate disease or promote disease progression.

Intestinal barrier dysfunction and increased intestinal permeability can be linked to various liver diseases including nonalcoholic fatty liver disease (NAFLD), nonalcoholic steatohepatitis (NASH), and cirrhosis (e.g., alcohol cirrhosis)

In an embodiment, the present invention provides for the treatment of a patient with NAFLD. NAFLD represents a spectrum of diseases occurring in the absence of alcohol abuse. It is characterized by the presence of steatosis (fat in the liver) and may represent a hepatic manifestation of the metabolic syndrome (including obesity, diabetes and hypertriglyceridemia). The severity of NAFLD ranges from the relatively benign isolated predominantly macrovesicular steatosis (i.e., nonalcoholic fatty liver or NAFL) to non-alcoholic steatohepatitis (NASH). NASH is characterized by the histologic presence of steatosis, cytological ballooning, scattered inflammation and pericellular fibrosis. Hepatic fibrosis resulting from NASH may progress to cirrhosis of the liver or liver failure, and in some instances may lead to hepatocellular carcinoma. In some embodiments, methods of the invention reduce, ameliorate, or eliminate one or more symptoms of NAFLD or NASH, including any of the symptoms described herein (e.g., liver cirrhosis or liver fibrosis). In some embodiments, method of the invention prevents or slows the progression of NAFLD or NASH to hepatocellular carcinoma.

Larazotide is a peptide agent that promotes tight junction integrity and reduced epithelial permeability, including in the gastrointestinal tract (GI). Larazotide has the amino acid sequence: Gly Gly Val Leu Val Gln Pro Gly (SEQ ID NO:1), and can be formulated for systemic or targeted release in portions of the GI (e.g., stomach, small intestine and/or large intestine). In some embodiments, the patient receives a larazotide derivative, for example, having one or more amino acid substitutions, deletions, and/or insertions with respect to SEQ ID NO:1. For example, the derivative may have 1, 2, 3, 4, or 5 amino acid modifications independently selected from deletions, insertions, and substitutions with respect to SEQ ID NO:1. Exemplary larazotide derivatives are described in U.S. Pat. Nos. 8,785,374, 8,957,032, and 9,279,807, which are hereby incorporated by reference in their entirety. In some embodiments, the derivative has one or more non-genetically encoded amino acids, or one or more D-amino acids. In some embodiments, the larazotide derivative is a retro-inverso larazotide peptide or derivative thereof. The term “larazotide” or “larazotide treatment” refers to treatment with larazotide or a derivative that promotes tight junction integrity and/or reduced epithelial permeability.

Larazotide has been shown in clinical trials to exhibit benefit at reducing intestinal disease symptoms, particularly at lower doses (e.g., 0.5 mg dose). See US 2016/0022760, which is hereby incorporated by reference in its entirety. Higher doses (e.g., 1 mg and 2 mg doses) showed an attenuation of activity, or no activity at all. It is believed that an aminopeptidase located within the brush borders of the lumen surface may create larazotide-derived fragments, including fragments missing N-terminal glycine residues. For example, the fragments GVLVQPG (SEQ ID NO:2) and VLVQPG (SEQ ID NO:3) are largely inactive as tight junction regulators. Moreover, when these two fragments are mixed with full length larazotide, activity is completely abolished. Local buildup of these inactive larazotide fragments (due to excessive larazotide) may in fact compete and block function of the peptide. This would explain clinical observations that low doses of larazotide work best by avoiding the reservoir of competing inactive fragments.

In some embodiments, a larazotide derivative is administered that exhibits resistance to exopeptidases, such as aminopeptidases, thus avoiding substantial accumulation of inactive peptide fragments. Exemplary modifications include amino acid substitutions at the N- and/or C-terminus to reduce exopeptidase digestion, extension of the N- and/or C-termini to delay exopeptidase digestion of the functional peptide, incorporation of D amino acids, as well as cyclization. Exemplary larazotide derivatives are disclosed in U.S. 62/634,536, filed Feb. 23, 2018, which is hereby incorporated by reference in its entirety.

In various embodiments, the peptide has at least one, at least two, at least three, at least four, at least five, at least six, at least seven, or at least eight D-amino acids. In an embodiment, each amino acid of the larazotide derivative (other than Gly) is a D-amino acid, and is optionally a retro-inverso peptide. A retro-inverso peptide contains the inverse amino acid sequence (e.g., GPQVLVGG, SEQ ID NO:4), with all non-glycine amino acids present as D-amino acids. Retro-inverso peptides maintain side chain topology similar to that of the original L-amino acid peptide, and render the peptide more resistant to proteolytic degradation. In some embodiments, the N-terminal Gly of the retro-inverso peptide is substituted with Ala, Leu, Ile, Val, or Allylgly. In these or other embodiments, one or both of the C-terminal Gly residues of the retro inverso peptide is/are substituted with an amino acid independently selected from Ala, Leu, Ile, Val, or Allylgly.

In other embodiments, the peptide having the amino acid sequence of SEQ ID NO:1 has one or two D-amino acids at the N- and optionally the C-terminus, with all other amino acids in the L configuration. In these embodiments, the N- and/or C-terminus are substituted or extended such that the peptide does not have a glycine at the terminus (Gly does not have D- and L-configurations). In some embodiments, the terminal Gly residues are replaced with an amino acid independently selected from D-Ala, D-Leu, D-Ile, D-Val, or D-Allylgly.

Larazotide (or derivative) may be administered in any suitable form, including as a salt. For example, larazotide may be administered as an acetate salt. Salts of larazotide, including the acetate salt and hydrochloride salt, are described in US 2013/0281384, which is hereby incorporated by reference in its entirety. Alternative salts may be employed, including any pharmaceutically acceptable salt of the peptide such as those listed in Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In various embodiments, the present invention provides pharmaceutical compositions comprising the larazotide or derivative in various formulations. Pharmaceutical composition can take the form of tablets, pills, pellets, capsules, capsules containing liquids, capsules containing multiparticulates, powders, solutions, emulsions, drops, suspensions, delayed-release formulations, sustained-release formulations, controlled-release formulations, or any other form suitable for use.

In various embodiments, the present invention provides pharmaceutical compositions comprising the larazotide or derivative that are co-formulated with other therapeutic agents for the treatment of hyperglycemia or diabetes mellitus and which are suitable for oral delivery. For example, in some embodiments, larazotide or derivative is co-formulated with a therapeutic agent selected from metformin, sulphonylurea, pPAR-gamma agonist, meglitinide, α-glucosidase inhibitors, thiazolidinediones, or combination thereof. In some embodiments, larazotide or derivative is co-formulated with a therapeutic agent selected from dulaglutide, semaglutide, liraglutide, metformin hydrochloride, glimepiride, gilbenclamide, glyburide, gilclazide, glipizide, tolbutamide, acarbose, miglitol, voglibose, pioglitazone, and rosiglitazone. In some embodiments, larazotide or derivative is co-formulated with a therapeutic agent, such as a dual GLP-1/GIP receptor agonist (e.g., LY3298176). In some embodiments, the larazotide or derivative is coformulated with a GLP-1 receptor agonist (e.g., dulaglutide or semaglutide) or basal insulin that is suitable or engineered for oral delivery. In some embodiments, the GLP-1 receptor agonist engineered for oral delivery is a GLP-1 peptide analog having an acylation of the peptide backbone that allows for binding to albumin (e.g., C18 fatty di-acid). In some embodiments, the GLP-1 peptide analog has one or more amino acid substitutions to reduce DPP-4 proteolysis, such as a substitution for Ala 8 (e.g., substitution with alpha-aminobutyric acid). The analog may have additional substitutions such as at position 34 (e.g., K34R).

In various embodiments, the present invention provides pharmaceutical compositions comprising the larazotide or derivative that are formulated as an injectable, optionally with other agents for treatment of hyperglycemia. Systemically administered larazotide may reduce permeability of endothelial tight junctions in the vasculature and/or endothelial and/or epithelial tight junctions of organs, including endothelial and epithelial tight junctions of the liver, kidneys, pancreas, etc. For example, such pharmaceutical compositions comprising the larazotide or derivative are suitable for parenteral administration, such as by subcutaneous, intramuscular, or intravenous administration. In some embodiments, the larazotide or derivative further comprises a half-life extension moiety. For example, the larazotide or derivative may contain one or more conjugated groups or fusion sequences that render the larazotide or derivative greater than ˜50 kDa to avoid renal filtration and/or allow for binding to serum proteins. Non-limiting examples of half-life extension moieties include fusion to albumin, Fc, transferrin, or elastin-like peptide amino acid sequences, or conjugation of one or more chemical groups such a PEG or fatty acid. In various embodiments, the present invention provides pharmaceutical compositions comprising the larazotide or derivative suitable for injection that are co-formulated with other therapeutic agents for the treatment of hyperglycemia or diabetes mellitus such as a GLP-1 receptor agonist (e.g., liraglutide, dulaglutide, or other GLP-1 or exendin-4 peptide analog) or basal or prandial insulin or a dual GLP-1/GIP receptor agonist or a GIP receptor antagonist.

The sustained or controlled release formulation, particularly for administration to the GI, may functionally release peptide over the course of at least about 2 hours, or at least about 2.5 hours, or at least about 3 hours, or at least about 4 hours, or at least about 5 hours. The term “functional release” refers to the release of larazotide such that the larazotide peptide can interact with cells of the intestinal epithelium to promote tight junction assembly. In various embodiments, larazotide is formulated as a plurality of particles that release larazotide at different times in intestinal fluid, or at different locations in the intestine. In other embodiments, the formulation releases larazotide in a form that provides for a local sustained release at one or more locations, including sustained release from particles, gels, emulsions, or biodegradable matrix. In some embodiments, the sustained or controlled release composition (e.g., comprising peptide-containing particles, gels, emulsions, or biodegradable matrix) begins to release peptide starting within about 15 or 30 minutes of exposure to simulated intestinal fluid, with release of peptide continuing for at least about 180 minutes, or at least about 210 minutes, or at least about 240 minutes, or at least about 280 minutes of exposure to simulated intestinal fluid. Release profiles can be prepared, for example, using particles with different enteric polymer coats and/or different thicknesses of the polymer coats. Exemplary particles are described herein.

The sustained release or controlled release formulation avoids accumulation of inactive fragments that may act as competitive inhibitors. For example, the formulation may deliver and/or functionally release from 0.5 to about 5 mg of larazotide or derivative, or from about 0.5 to about 4 mg of larazotide or derivative, or from about 0.5 to about 3 mg of larazotide or derivative, or from about 0.5 to about 2 mg of larazotide or derivative, or from about 0.5 to about 1 mg of larazotide or derivative. In various embodiments, the sustained release or controlled release formulation contains at least 1 mg or at least 2 mg of larazotide or derivative. For example, the formulation may contain from about 1 mg to about 5 mg of larazotide or derivative, or about 1 mg to about 3 mg of larazotide or derivative.

In some embodiments, the formulation for delivery to the GI may comprise or deliver and/or functionally release from about 0.1 mg to about 1 mg of larazotide or derivative, or from about 0.1 mg to about 0.8 mg of larazotide or derivative, or from about 0.1 mg to about 0.6 mg of larazotide or derivative, or from about 0.1 mg to about 0.4 mg of larazotide or derivative, or from about 0.1 mg to about 0.5 mg of larazotide or derivative, or from about 0.5 mg to 1 mg of larazotide or derivative. In various embodiments, the sustained release or controlled release formulation contains at least 0.1 mg or at least 1 mg of larazotide or derivative. For example, the formulation may contain from about 0.1 mg to about 1 mg of larazotide or derivative, or about 0.5 mg to about 1 mg of larazotide or derivative.

In one embodiment, the composition comprising peptide remains essentially intact, or may be essentially insoluble, in gastric fluid. The stability of a gastric-resistant coating can be pH dependent. Delayed-release coatings that are pH dependent will be substantially stable in acidic environments (pH 5 or less), and substantially unstable in near neutral to alkaline environments (pH greater than 5). For example, a delayed-release coating can be employed that will essentially disintegrate or dissolve in near neutral to alkaline environments such as are found in the small intestine. Examples of simulated gastric fluid and simulated intestinal fluid include, but are not limited to, those disclosed in the 2005 Pharmacopeia 23NF/28USP in Test Solutions and/or other simulated gastric fluids and simulated intestinal fluids known to those of skill in the art, for example, simulated gastric fluid and/or intestinal fluid prepared without enzymes.

Alternatively, the stability of the delayed-release coating can be enzyme-dependent. Delayed-release coatings that are enzyme dependent will be substantially stable in fluid that does not contain a particular enzyme and substantially unstable in fluid containing the enzyme. The delayed-release coating will essentially disintegrate or dissolve in fluid containing the appropriate enzyme. Enzyme-dependent control can be brought about, for example, by using materials which release the active ingredient only on exposure to enzymes in the intestine, such as galactomannans.

In some embodiments, the formulation releases larazotide or derivative in simulated gastric fluid, so as to release larazotide or derivative in the stomach. For example, a population of peptide-coated particles can be employed having an outer coating that disintegrates in simulated gastric fluid.

Various formulations can be employed to deliver the larazotide or derivative to a location of interest. For example, the compositions may be formulated for targeted delivery to the gastrointestinal tract including the stomach, small intestine, large intestine and rectum including all subsections thereof. By targeting release of larazotide or derivative in the affected region(s) (e.g. stomach, duodenum, jejunum, ileum, colon transversum, colon descendens, colon ascendens, colon sigmoidenum and cecum), tight junction integrity at various portions of the GI can be improved.

In some embodiments, the composition is formulated to release in the small intestine, including one or more of the duodenum, jejunum, and/or the ileum, and optionally the stomach. Alternatively, or in addition, the composition is formulated to release in the large intestine, including one or more of the cecum, the ascending colon, the transverse colon, the descending colon, and/or the sigmoid colon.

In various embodiments, the composition may be formulated to have sustained-release profiles, i.e. slow release of the larazotide in the GI tract over an extended period of time. In various embodiments, the composition may be formulated to have a delayed-release profile, i.e. not immediately release the larazotide upon ingestion; rather, postponement of the release until the composition is lower in the gastrointestinal tract; for example, for release in the small intestine (e.g., one or more of duodenum, jejunum, and ileum) and/or the large intestine (e.g., one or more of cecum, ascending, transverse, descending or sigmoid portions of the colon, and rectum). In an embodiment, the pharmaceutical composition is formulated to have a delayed-release profile as described in, for example, U.S. Pat. No. 8,168,594, the entire contents of which are hereby incorporated by reference.

For example, the larazotide or derivative may be administered to the duodenum of the patient, as an oral dosage, delayed-release composition that contains larazotide (or derivative)-coated beads that are stable in gastric fluid and unstable in intestinal fluid so as to substantially release the peptide in the duodenum. The composition may further comprise a second population of beads with a pH-dependent coating to affect release of the peptide in the jejunum of the patient. For example, the second population of beads may release the larazotide or derivative about 30 minutes after the beads releasing peptide in the duodenum (based on release in simulated intestinal fluid). The composition may further comprise a third population of beads with a pH-dependent coating to affect release of the peptide in the ileum of the patient. For example, the third population of beads may release the larazotide or derivative at least about 30 minutes after the beads releasing peptide in the jejunum (based on release in simulated intestinal fluid). The oral dosage composition can be in the form of a capsule or tablet. The pH-dependent coating in some embodiments is a 1:1 co-polymer of methacrylic acid and ethyl acrylate, wherein the thickness of the layer determines the release profile of each bead. In these or other embodiments, the formulation comprises a population of beads that release larazotide or derivative in the stomach, i.e., release larazotide or derivative when exposed to simulated gastric fluid. The beads may have one or more additional coatings such as a base coat, a separating layer, and an overcoat layer.

In an exemplary oral dosage composition, an effective amount of larazotide (e.g., as the acetate salt) is provided in first delayed-release particles that are capable of releasing larazotide or derivative in the duodenum of a patient, and second delayed release particles that are capable of releasing larazotide or derivative in the jejunum of a patient, and optionally a third delayed release particle capable of releasing larazotide or derivative in the stomach and/or ileum of a patient. Each particle may have a core particle, a coat comprising larazotide or derivative over the core particle, and a delayed-release coating (e.g., a 1:1 co-polymer of acrylate and methacrylate) outside the coat comprising larazotide or derivative. Whereas the first delayed-release particles release at least 70% of the larazotide or derivative in the first delayed-release particles by about 60 minutes of exposure to simulated intestinal fluid having a pH of greater than 5; the second delayed-release particles release at least 70% of the larazotide or derivative by about 30 and about 90 minutes of exposure to simulated intestinal fluid having a pH of greater than 5. The third delayed-release particles (for release in the ileum) release at least 70% of the larazotide or derivative by about 120 minutes to about 240 minutes (e.g., about 120 minutes to about 180 minutes) of exposure to simulated intestinal fluid. For release in the stomach, peptide will release in the presence of simulated gastric fluid.

In some embodiments where the damage to the colon is involved, the larazotide or derivative may be administered to the colon of a patient, as an oral dosage, modified-release composition. Various colon-specific delivery approaches may be utilized. For example, the modified release formulation may be formulated using a colon-specific drug delivery system (CODES) as described for example, in Li et al., AAPS PharmSciTech (2002), 3(4): 1-9, the entire contents of which are incorporated herein by reference. Drug release in such a system is triggered by colonic microflora coupled with pH-sensitive polymer coatings. For example, the formulation may be designed as a core tablet with three layers of polymer. The first coating is an acid-soluble polymer (e.g., EUDRAGIT E), the outer coating is enteric, along with a hydroxypropyl methylcellulose barrier layer interposed in between. In another embodiment, colon delivery may be achieved by formulating the larazotide or derivative with specific polymers that degrade in the colon such as, for example, pectin. The pectin may be further gelled or crosslinked with a cation such as a zinc cation. Additional colon specific formulations include, but are not limited to, pressure-controlled drug delivery systems (prepared with, for example, ethylcellulose) and osmotic controlled drug delivery systems (i.e., ORDS-CT).

In some embodiments, the delayed-release coating (including for sustained release and controlled release formulations) includes an enteric agent that is substantially stable in acidic environments and substantially unstable in near neutral to alkaline environments. In an embodiment, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymer include, for example, EUDRAGIT® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12,5, and S 12,5 P. In some embodiments, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 and S 12,5 P is used. The enteric agent may be a combination of the foregoing solutions or dispersions.

In another embodiment, the delayed-release coating may degrade as a function of time when in aqueous solution without regard to the pH and/or presence of enzymes in the solution. Such a coating may comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings may be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, EUDRAGIT NE®, polyvinyl esters, polyvinyl acetals, polyacrylic acid esters, butadiene styrene copolymers, and the like.

The present invention also provides for compositions that release multiple doses of the larazotide or derivative along the gastrointestinal tract. For example, the composition and/or formulation can release multiple doses of the larazotide or derivative at different locations along the intestines, at different times, and/or at different pH. The overall release profile of such a formulation may be adjusted using, for example, multiple particle types or multiple layers. For example, in one embodiment, a first dose of the larazotide or derivative may be formulated for release in, for example, the small intestine (e.g., one or more of duodenum, jejunum, ileum), whereas a second dose is formulated for delayed release in, for example, the large intestines (e.g., one or more of cecum, ascending, transverse, descending or sigmoid portions of the colon, and rectum). In various embodiments, the composition and/or formulation may release at least three doses, at least four doses, or at least five doses of the larazotide or derivative at different locations along the intestines, at different times, and/or at different pH. In these embodiments, a dose is typically from about 0.1 mg to about 1 mg of larazotide or derivative, or from about 0.1 mg to about 0.8 mg of larazotide or derivative, or from about 0.1 mg to about 0.6 mg of larazotide or derivative, or from about 0.1 mg to about 0.4 mg of larazotide or derivative, or from about 0.1 mg to about 0.5 mg of larazotide or derivative, or from about 0.5 mg to 1 mg of larazotide or derivative.

In accordance with certain embodiments of the invention, larazotide or derivative is administered more than once daily to promote GI tight junction integrity. For example, larazotide or derivative may be administered about two times daily, about three times daily, about four times daily, or about five times daily.

In some embodiments, the subject further receives a probiotic. Probiotics suitable for use in the present invention include, but are not limited to, Saccharomyces boulardii; Lactobacillus rhamnosus GG; Lactobacillus plantarum 299v; Clostridium butyricum M588; Clostridium difficile VP20621 (non-toxigenic C. difficile strain); combination of Lactobacillus casei, Lactobacillus acidophilus (Bio-K+CL1285); combination of Lactobacillus casei, Lactobacillus bulgaricus, Streptococcus thermophilus (Actimel); combination of Lactobacillus acidophilus, Bifidobacterium bifidum (Florajen3); combination of Lactobacillus acidophilus, Lactobacillus bulgaricus delbrueckii subsp. bulgaricus, Lactobacillus bulgaricus casei, Lactobacillus bulgaricus plantarum, Bifidobacterium longum, Bifidobacterium infantis, Bifidobacterium breve, and Streptococcus salivarius subsp. thermophilus (VSL #3)).

In accordance with embodiments of the invention, larazotide or a derivative thereof may be heterologously expressed in a microorganism that is a commensal microorganism of the human gastrointestinal tract, or a microbial species that find conventional use as a probiotic, as described in PCT/US19/19348, which is hereby incorporated by reference in its entirety. For example, the microorganism may be a bacterium or fungus, and exemplary microorganisms include those of the genus Saccharomyces, Lactobacillus, Clostridium, Streptococcus, Staphylococcus, or Bifidobacterium. For example, the microorganism may be a species selected from Saccharomyces boulardii, Lactobacillus rhamnosus, Lactobacillus plantarum, Clostridium butyricum, non-toxigenic Clostridium difficile, Lactobacillus casei, Lactobacillus acidophilus, Lactobacillus bulgaricus, Streptococcus thermophilus, Bifidobacterium bifidum, Bifidobacterium longum, Bifidobacterium lactis, Bifidobacterium infantis, Bifidobacterium breve, and Streptococcus salivarius. In some embodiments, a probiotic strain (bacterial or fungal) is engineered for expression and optionally secretion of larazotide or derivative thereof from the cell. In some embodiments, a bacteriophage comprising a gene encoding larazotide under control of a bacterial promoter is administered to the patient. The bacteriophage may be lytic or lysogenic.

In various embodiments, the microorganism is derived from a commensal microorganism of the human gastrointestinal tract, such as those of the genera Bacteroides, Faecalibacterium, Corynebacterium, Eubacterium, Ruminococcus, Peptococcus, Peptostreptococcus, Escherichia, or Helicobacter. In some embodiments, the microbe is E. coli. In some embodiments, the microbe is selected from a Fungal genera of Candida, Saccharomyces, Aspergillus, Penicillium, Rhodotorula, Trametes, Pleospora, Sclerotinia, Bullera, and Galactomyces.

Where larazotide or derivative is delivered as a probiotic, the probiotic may be delivered, for example, no more than about once daily, no more than about once weekly, or no more than about once monthly.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

1. A method for treating hyperglycemia in a subject, comprising administering a pharmaceutical composition comprising an effective amount of larazotide in a regimen sufficient to reduce intestinal barrier dysfunction.

2. The method of claim 1, wherein the patient has diabetes mellitus type 1 or type 2.

3. The method of claim 1, wherein the patient has metabolic syndrome.

4. The method of claim 3, wherein the patient is obese.

5. The method of claim 1, wherein the patient is prediabetic.

6. The method of any one of claims 1 to 5, wherein the patient is undergoing therapy with metformin, basal insulin, GLP-1 receptor agonist, dual GIP/GLP-1 receptor agonist, GIP receptor antagonist, sulphonylurea, meglitinide, pPAR-gamma agonist, α-glucosidase inhibitor, or thiazolidinedione.

7. The method of claim 6, wherein the patient is undergoing therapy with a GLP-1 receptor agonist optionally selected from liraglutide, semaglutide, and dulaglutide.

8. The method of claim 6 or 7, wherein the patient remains hyperglycemic despite therapy with metformin, basal insulin, GLP-1 receptor agonist, dual GIP/GLP-1 receptor agonist, GIP receptor antagonist, sulphonylurea, meglitinide, pPAR-gamma agonist, α-glucosidase inhibitor, or a thiazolidinedione.

9. The method of any one of claims 1 to 8, wherein the patient further has an inflammatory liver disease or fatty liver disease.

10. The method of claim 9, wherein the patient has non-alcoholic fatty acid liver disease (NAFLD), non-alcoholic steatohepatitis (NASH), or hepatitis.

11. The method of any one of claims 1 to 8, wherein the patient further has impaired kidney function.

12. The method of claim 11, wherein the patient has chronic kidney disease or diabetic nephropathy.

13. The method of any one of claims 1 to 12, wherein larazotide is administered to the patient.

14. The method of any one of claims 1 to 12, wherein a larazotide derivative is administered to the patient.

15. The method of claim 14, wherein the larazotide derivative has from 1 to 5 amino acid modifications independently selected from deletions, insertions, and substitutions with respect to SEQ ID NO:1.

16. The method of claim 14 or 15, wherein the derivative has one or more non-genetically encoded amino acids or one or more D-amino acids.

17. The method of claim 14, wherein the derivative is a retro-inverso larazotide peptide or derivative thereof.

18. The method of any one of claims 1 to 17, wherein the larazotide or derivative is administered as a salt.

19. The method of any one of claims 1 to 18, wherein larazotide or derivative is co-formulated or administered with an orally administrable therapeutic agent for the treatment of hyperglycemia or diabetes mellitus.

20. The method of claim 19, wherein the therapeutic agent is GLP-1 receptor agonist, dual GIP/GLP-1 receptor agonist, GIP receptor antagonist, metformin, sulphonylurea, pPAR-gamma agonist, meglitinide, α-glucosidase inhibitor, insulin, or thiazolidinedione.

21. The method of claim 20, wherein the larazotide or derivative is co-formulated with semaglutide.

22. The method of any one of claims 1 to 21, wherein the pharmaceutical composition comprising larazotide releases larazotide or derivative over the course of at least about 2 hours.

23. The method of claim 22, wherein the pharmaceutical composition begins to release larazotide or derivative starting within about 15 or 30 minutes of exposure to simulated intestinal fluid, with release of peptide continuing for at least about 180 minutes.

24. The method of claim 22 or 23, wherein the formulation releases from 0.5 to about 5 mg of larazotide or derivative.

25. The method of claim 24, wherein the formulation releases from about 0.5 to about 1 mg of larazotide or derivative.

26. The method of any one of claims 1 to 25, wherein the composition is formulated to release larazotide or derivative in the small intestine, including one or more of the duodenum, jejunum, and/or the ileum.

27. The method of claim 26, wherein the composition is further formulated to release larazotide or derivative in the large intestine, including one or more of the cecum, the ascending colon, the transverse colon, the descending colon, and/or the sigmoid colon.

28. The method of any one of claims 1 to 27, wherein the larazotide or derivative is administered more than once daily.

29. The method of any one of claims 1 to 15, wherein the larazotide or a derivative is heterologously expressed in a microorganism.

30. A pharmaceutical composition comprising an effective amount of larazotide or a derivative and a therapeutic agent selected from GLP-1 receptor agonist, dual GIP/GLP-1 receptor agonist, GIP receptor antagonist, metformin, sulphonylurea, pPAR-gamma agonist, meglitinide, α-glucosidase inhibitor, insulin, and thiazolidinedione.

31. The pharmaceutical composition of claim 30, comprising larazotide.

32. The pharmaceutical composition of claim 30, comprising a larazotide derivative.

33. The pharmaceutical composition of claim 32, wherein the larazotide derivative has from 1 to 5 amino acid modifications independently selected from deletions, insertions, and substitutions with respect to SEQ ID NO:1.

34. The pharmaceutical composition of claim 32 or 33, wherein the derivative has one or more non-genetically encoded amino acids or one or more D-amino acids.

35. The pharmaceutical composition of claim 32, wherein the derivative is a retro-inverso larazotide peptide or derivative thereof.

36. The pharmaceutical composition of any one of claims 30 to 35, wherein the larazotide or derivative is in the form of a salt.

37. The pharmaceutical composition of any one of claims 30 to 36, wherein the composition is formulated for oral administration.

38. The pharmaceutical composition of claim 37, wherein the larazotide or derivative is formulated with a GLP-1 receptor agonist.

39. The pharmaceutical composition of claim 38, wherein the GLP-1 receptor agonist is semaglutide.

40. The pharmaceutical composition of any one of claims 30 to 39, wherein larazotide or derivative is released over the course of at least about 2 hours in simulated intestinal fluid.

41. The pharmaceutical composition of claim 40, wherein the pharmaceutical composition begins to release larazotide or derivative starting within about 15 or 30 minutes of exposure to simulated intestinal fluid, with release of larazotide or derivative continuing for at least about 180 minutes.

42. The pharmaceutical composition of any one of claims 30 to 41, wherein the effective amount is from 0.5 to about 5 mg of larazotide or derivative.

43. The pharmaceutical composition of claim 42, wherein the effective amount is from about 0.5 to about 1 mg of larazotide or derivative.

44. The pharmaceutical composition of any one of claims 30 to 43, wherein the composition is formulated to release larazotide or derivative in the small intestine, including one or more of the duodenum, jejunum, and/or the ileum.

45. The pharmaceutical composition of claim 44, wherein the composition is further formulated to release in the large intestine, including one or more of the cecum, the ascending colon, the transverse colon, the descending colon, and/or the sigmoid colon.

46. The pharmaceutical composition of any one of claims 30 to 36, wherein the composition is formulated for parenteral administration.

47. The pharmaceutical composition of claim 46, wherein the composition is formulated for subcutaneous, intramuscular, or intravenous administration.

48. The pharmaceutical composition of claim 46 or 47, wherein the larazotide or derivative further comprises a half-life extension moiety.

49. The pharmaceutical composition of claim 48, wherein the larazotide or derivative comprises one or more conjugated groups or fusion sequences that render the larazotide or derivative greater than ˜50 kDa and/or allow for binding to serum proteins.

50. The pharmaceutical composition of claim 49, wherein the half-life extension moiety is a fusion to albumin, Fc, transferrin, or elastin-like peptide amino acid sequences.

51. The pharmaceutical composition of claim 49, wherein the half-life extension moiety is one or more polyethylene glycol (PEG) groups or fatty acids.

52. The pharmaceutical composition of any one of claims 46 to 51, wherein the larazotide or derivative is formulated with a GLP-1 receptor agonist, or with basal or prandial insulin.

53. The pharmaceutical composition of claim 52, wherein the larazotide or derivative is co-formulated with liraglutide or dulaglutide.

54. A method for treating hyperglycemia in a subject, comprising administering a pharmaceutical composition of any one of claims 30 to 53.

55. The method of claim 54, wherein the patient has diabetes mellitus type 1 or type 2.

56. The method of claim 54 or 55, wherein the patient has metabolic syndrome.

57. The method of claim 56, wherein the patient is obese.

58. The method of claim 54, wherein the patient is prediabetic.

59. The method of any of claims 54 to 58, wherein the patient further has an inflammatory liver disease, fatty liver disease, and/or kidney disease.

60. The method of claim 59, wherein the patient has non-alcoholic fatty acid liver disease (NAFLD), non-alcoholic steatohepatitis (NASH), and/or chronic kidney disease (CKD).

61. The method of any one of claims 54 to 60, wherein the patient exhibits pancreatitis, adipose tissue inflammation, atherosclerosis, and/or neurodegeneration.


SEQUENCE LISTING (4 sequences)

SEQ ID NO: 1 - protein, 8 aa, Artificial Sequence
  Synthetic polypeptide
GGVLVQPG

SEQ ID NO: 2 - protein, 7 aa, Artificial Sequence
  Synthetic polypeptide
GVLVQPG

SEQ ID NO: 3 - protein, 6 aa, Artificial Sequence
  Synthetic polypeptide
VLVQPG

SEQ ID NO: 4 - protein, 8 aa, Artificial Sequence
  Synthetic polypeptide
GPQVLVGG